(19) United States
(12) Patent Application Publication
(10) Pub. No.: US 2009/0082205 A1
(43) Pub. Date: Mar. 26, 2009

(54) BIOLOGICAL COMPOSITION FOR GENERATING AND FEEDING MICROORGANISMS THAT ARE INTENDED FOR DISTRIBUTION IN AN AGRICULTURAL SYSTEM

(76) Inventor: Raymond W. Stock, South Jordan, UT (US)

Correspondence Address: ADVANTIA LAW GROUP, 9035 SOUTH 1300 EAST, SUITE 200, SANDY, UT, 84094, US

(21) Appl. No.: 12/259,175

(22) Filed: Oct. 27, 2008

Related U.S. Application Data
(63) Continuation-in-part of application No. 11/036,422, filed on Jan. 13, 2005, now abandoned.
(60) Provisional application No. 60/566,893, filed on Apr. 29, 2004.

(51) Int. Cl. A01N 63/00; C12N 1/00; C12N 1/20; A01P 21/00; C12N 1/16

(52) U.S. Cl. 504/117; 435/243; 435/252.5; 435/253.3; 435/252.1; 435/255.1

(57) ABSTRACT

There is a biological composition and method of use for generating and feeding microorganisms that are intended for distribution in an agricultural system. Included is a micro-organism quantity, and a nutrient quantity which may have a supplement, a short-term nutrient and a long term nutrient. The composition may be applied to a container having water wherein the microorganisms rapidly reproduce. As the microorganisms reproduce they may be extracted. Once extracted, they may be introduced into an ecosystem, such as being injected into an irrigation system and being dispersed over crops and crop soil. There, the microorganisms may enhance vegetation growth, health, production, strength and efficiency by producing cellulase enzymes, converting nutrients to a form usable by vegetation, loosening soil, enhancing water retention in soil, and/or combating parasitic organisms.

This invention is a Continuation-In-Part Application and claims priority under 35 U.S.C. § 120, to U.S. Non-Provisional application Ser. No. 11/036,422, to Raymond W. Stock, filed on Jan. 13, 2005, which is incorporated by reference herein.

### BACKGROUND OF THE INVENTION

1. Field of the Invention

The present invention relates to biological compositions, specifically to biological compositions for generating and feeding microorganisms that are intended for distribution in an agricultural system for the purposes of promoting and facilitating healthy growth and consistent harvests.

2. Description of the Related Art

The agriculture industry is an important component of society because it is responsible for producing food for human and animal consumption. In furtherance of this responsibility, the agricultural industry relies heavily on the soil from which crops are grown and cultivated. Over time, the repeated use of soil for growing crops can result in a reduction of the available nutrients and nutrient-producing soil bacteria. As this occurs, plants can begin to show signs of inadequate nutrition by having stunted growth and/or poor health characteristics. To overcome the problems of nutrient depleted soil, farmers have been adding biological and/or nutrient-containing substances to their soil, often in the form of solid particles of such materials distributed over or turned into the soil.

In some systems, liquid fertilizer may be distributed through an irrigation system. However, microorganisms are more difficult to work with than fertilizer as they require tighter environmental constraints, are more difficult to ship, manufacture, and distribute. Accordingly, microorganisms are generally produced and applied to soil in a solid/granular form such as coupled to pellets, clods, grains, or dust. Accordingly, a farmer may purchase a quantity of microorganisms in such a form and then distribute those over or in the soil as desired. Results are dependent on the amount purchased, the conditions of the environment during and after distribution, the micro-flora/fauna already present in the system, and etc. In practice, farmers have seen limited benefits that are comparable to the costs. Further, results tend to be proportional to amounts purchased and distributed.

Accordingly, growing microorganisms for use in agriculture has been generally marginally successful because results are generally proportional to and equal to costs. As such, improvements in growing and feeding micro-organism rich compositions used to condition soil and the manufacturing and methods thereof continue to be sought. Some improvements have been made in the field. Examples include, but are not limited to, the following references, which are incorporated by reference herein for their supporting teachings:

U.S. Pat. No. 5,447,866, issued to Runyon, discloses a single, unitary reactor and its feed mechanism for microorganisms, nutrients, and, commonly, also enzymes are disclosed. The reactor forms and dispenses a slurried biomass for turf and soil enhancement, water body treatment and agricultural application through conventional irrigation systems. The feed mechanism feed the various components into the slurry within the reactor on any desired schedule, including different schedules for different components. The microorganisms, nutrients and enzymes are provided in the form of pellets to individual compartments within the feed device, and they are individually injected into the slurry intermittently upon a predetermined schedule. Preferably the housing will contain a plurality of the compartments for a variety of different slurry components. The component pellets may be of any convenient shape and size, but preferably will be either disk-shaped or spherical.

U.S. Pat. No. 5,739,031, issued to Runyon, discloses a simplified method is disclosed for handling growth and dispersion of microorganisms in solution and which is adaptable to many different end-use applications, including treatment of turf, decorative and ornamental vegetation, horticultural plants and agricultural crops. Provision of the initial micro-organism materials in the form of aqueous suspensions which are incorporated in liquid form into a large volume of water in a vessel and subsequent growth of biomass in the vessel provides for simplicity and flexibility not previously obtained from the prior art devices and methods. The method provides biomasses containing microorganisms to enhance vegetation growth, provide disease- and/or pest resistance, detoxification, solids removal, or any combination thereof. Apparatus to carry out the method of the invention is also disclosed, includes efficient and simply liquid feed devices.

U.S. Pat. No. 5,227,068, issued to Runyon, discloses a method and apparatus are described for providing improved irrigation to vegetation in open areas such as parks and golf courses, including providing improved microbiotic and nutrient supplies to such vegetation in a non-sterilized environment. In its method aspects, the invention comprises combining in an aqueous slurry effective amounts of first and second microorganisms, enzymes and nutrients, allowing the combined microorganisms to grow in a closed reservoir to reach levels of concentrations of the first and second microorganisms effective for irrigation, enhancement and nutrition of vegetation, and then applying the concentrated materials to the vegetation in a water slurry in quantities sufficient to effectively irrigate and provide nutrition to the vegetation and to provide it with protection against harmful microorganisms. Apparatus is described for the performance of the method, and includes an enhancement vessel adapted to retain the combined materials for a time sufficient for the first and second microorganisms to increase in concentration to provide a significant degree of nutrition and protection against harmful microorganisms to vegetation to which they are applied; and a conduit to convey the slurry to the vegetation and to apply it to the vegetation in effective quantities. Typical of the applications for which this invention can be used is the irrigation, protection and nutrition of vegetation for golf course, parks, and other landscaped areas, to enhance the growth and physical condition of the vegetation of the course or park.

U.S. Pat. No. 6,335,191, issued to Kiplinger et al., discloses an automated system and method is provided for cultivating bacteria in a fluid medium and thereafter selectively discharging the fluid medium, wherein an initial supply of the selected strain or strains of bacteria is combined with nutrients and water in a biogenerator in the presence of air to promote mixing and bacterial cultivation. The system and method utilize a vortex created by recirculation of the fluid medium to achieve aeration and mixing without substantial foaming. The system and method are particularly useful for supplying bacteria to control grease accumulation in restaurant grease traps. The system and method use a biogeneration chamber, which has a cylindrical sidewall and surface on the inner side. Further, the chamber has a top and a conical bottom. The top has inlet ports and a vent port. There is also a outlet port in the conical bottom. The conical bottom also has an orifice and recirculated fluid inlet port that is directed tangentially along the inside surface of the sidewall to create a downwardly spiraling vortex in the biogeneration chamber.

U.S. Pat. No. 7,081,361, issued to Peace et al., discloses a biomass generator useful for continuously growing and withdrawing bacteria to be used in a desired beneficial application, the generator having a bacteria growth chamber; water and nutrient inlet ports; upper and lower outlet ports; a recirculating pump withdrawing and reintroducing fluid to establish a vortex in the growth chamber while controlling foaming and pump cavitation; a low pressure air inlet line discharging air inside the growth chamber above the vortex; a fluid discharge line receiving bacteria-containing fluid from the upper outlet port of the growth chamber; a flush line discharging wash water into the fluid discharge line; and an electrical controller cooperating with solenoid-operated valves and a feeder mechanism to periodically introduce water and nutrients into the chamber, thereby simultaneously causing bacteria-containing fluid to be discharged from the growth chamber through the upper outlet port. A method for growing bacteria using the subject apparatus is also disclosed.

While microorganisms feeding and growing mechanisms and methods have been developed and used for many years, the results have been less than optimal. In particular, systems, compositions, devices and methods sometimes fail to provide sufficient nutrients for micro-organism growth and reproduction, are too expensive to operate/maintain, fail to cause microorganisms to reproduce at a satisfactory rate, fail to facilitate growth of the appropriate microorganisms, have short storage lifetimes, are difficult/expensive to manufacture, are difficult/expensive to ship, and require too much upkeep and/or decision-making on the part of system operators. Additionally, some substances are difficult to apply because of their nature to clump or otherwise not be spread and nourished evenly. Further, nutrient quantities are often provided to microorganisms in amounts insufficient for growth and reproduction of the microorganisms. Accordingly, this can cause the soil to be over-fertilized in some areas and under-fertilized in others, which results in overall poor growing conditions.

What is needed is a biological composition and method for generating and feeding microorganisms that are intended for distribution in an agricultural system that solves one or more of the problems described herein or that would come to the attention of one skilled in the art upon reading this specification.

### SUMMARY OF THE INVENTION

The present invention has been developed in response to the present state of the art, and in particular, in response to the problems and needs in the art that have not yet been fully solved. Accordingly, the present invention has been developed to provide a biological composition and methods of use thereof for generating and feeding microorganisms intended for use in ecosystems, such as, conditioning soil for use by crops.

In one embodiment, there is a biological composition for generating and feeding microorganisms that are intended for distribution in an agricultural system. The biological composition may include a micro-organism quantity and a nutrient quantity. The micro-organism quantity may have at least a first generation of microorganisms characterized by an ability to enhance vegetation growth. The nutrient quantity may be included in an amount sufficient to sustain reproduction of the micro-organism quantity for a reproduction period. The nutrient quantity may include a supplement quantity, a short-term nutrient quantity and a long-term nutrient quantity. The supplement quantity may be included in an amount sufficient to nourish the micro-organism quantity for the reproduction period. The short-term nutrient quantity may be included in an amount sufficient to sustain initial reproduction of at least the first generation of microorganisms and at least a subsequent generation of microorganisms for an initial portion of the reproduction period. The long-term nutrient quantity may have a longer period of consumption by the micro-organism quantity than the short-term nutrient quantity. The long-term nutrient quantity may be included in an amount sufficient to sustain continued reproduction of at least a continuing generation microorganisms and until the expiration of the reproduction period.

In another embodiment, the micro-organism quantity may be selected from the group consisting of bacillus subtilis, bacillus lichenformis, bacillus cereus, bacillus megaterium, fluorescent pseudomonas, azobacter, cellulase enzyme producing bacteria, yeasts, sub-cultures thereof, and combinations thereof.

In one embodiment, the supplement quantity is selected from the group consisting of vitamins; inorganic salts, amino acids, growth media, minerals, humate, humic acids, enzymes, chelating agents, complexing agents, sequestering agents, and combinations thereof.

In another embodiment, the short-term nutrient quantity may be selected from the group consisting of hydrolyzed collagen, bone meal, blood meal, carbon skeleton molecules, sugars, carbohydrates, folvic acid, organic acid, soy protein, peptone treated biological matter, and combinations thereof.

In one embodiment, the long-term nutrient quantity may be selected from the group consisting of wheat starch, soy flour, molasses, processed or raw animal and/or plant matter, and combinations thereof.

In another embodiment, the micro-organism quantity may be from about 5% to about 25% of the biological composition by volume. The nutrient quantity may be from 75% to 95% of the biological composition by volume. The supplement quantity may be from more than 0% to about 5%. The short-term nutrient quantity may be from about 50% to about 75%. The long-term nutrient quantity may be from about 15% to about 35% of the biological composition by volume.

In one embodiment, the reproduction period may be chosen any time from about two days to about one month and the initial portion of the reproduction period is from about 10% to about 70% of the reproduction period.

In another embodiment, there may be a method of generating and feeding microorganisms that are intended for distribution in an agricultural system, which method may include providing a container; providing a liquid mixture within the container, wherein the liquid mixture includes a water quantity, a micro-organism quantity, characterized by an ability to participate beneficially in a plant ecosystem, and a nutrient quantity; introducing oxygen into the liquid mixture; and circulating the liquid mixture.

In one embodiment, the nutrient quantity may include a short-term nutrient quantity and a long-term nutrient quantity. There may also be a supplement quantity included in the nutrient quantity.

In another embodiment, the method may further include extracting liquid mixture portions from the container at an average rate not significantly greater than an average regenerative rate of the liquid mixture.

In one embodiment, there may be a method of generating and feeding microorganisms that are intended for distribution in an agricultural system. This method may include providing a container; providing a liquid mixture within the container, wherein the liquid mixture includes a water quantity, a micro-organism quantity characterized by the ability to participate beneficially in the plant ecosystem, and a nutrient quantity; introducing oxygen into the liquid mixture; circulating the liquid mixture; extracting liquid mixture portions from the container; introducing the liquid mixture portions into irrigation water in an irrigation system configured to irrigate plants; and irrigating plants with the combined irrigation water and liquid mixture portions.

In another embodiment, the liquid mixture portions may be extracted from the container at an average rate not significantly greater than an average regenerative rate of the liquid mixture. Also, the liquid mixture portions may be extracted from the container from an extraction location disposed in a lower half portion of the container.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment, is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order for the advantages of the invention to be readily understood, a more particular description of the invention briefly described above will be rendered by reference to specific embodiments that are illustrated in the appended drawings. Understanding that these drawings depict only typical embodiments of the invention and are not, therefore, considered to be limiting of its scope, the invention will be described and explained with additional specificity and detail through the use of the accompanying drawings, in which:

FIGS. 1 and 2 illustrate a system configured to introduce microorganisms into a plant ecosystem according to one embodiment of the invention.

### DETAILED DESCRIPTION OF THE INVENTION

For the purposes of promoting an understanding of the principles of the invention, reference will now be made to the exemplary embodiments illustrated in the drawings, and specific language will be used to describe the same. It will, nevertheless, be understood, that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “one embodiment,” “an embodiment,” and similar language throughout this specification may, but do not necessarily, all refer to the same embodiment, different embodiments, or quantity parts of the same or different illustrated invention. Additionally, reference to the wording “an embodiment,” or the like, for two or more features, elements, etc. does not mean that the features are related, dissimilar, the same, etc. The use of the term “an embodiment,” or similar wording, is merely a convenient phrase to indicate optional features, which may or may not be part of the invention as claimed.

Each statement of an embodiment is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The independent embodiments are considered able to be combined in whole or in part one with another as the claims and/or art may direct, either directly or indirectly, implicitly or explicitly.

Finally, the fact that the wording “an embodiment,” or the like, does not appear at the beginning of every sentence in the specification, such as is the practice of some practitioners, is merely a convenience for the reader's clarity. However, it is the intention of this application to incorporate, by reference, the phrasing “an embodiment,” and the like, at or near the beginning of every sentence herein where logically possible and appropriate.

Within this specification, including the claims, the phrase “first generation of microorganisms,” and the like, is defined to include the entire set of microorganisms initially present in the biological composition when the biological composition is first put to use as described in the specification, including those that may be reproductive descendants of others present within the biological composition at the same time.

Within this specification, including the claims, the phrase “subsequent generation of microorganisms,” and the like, is defined to include a generation of microorganisms which are reproductive descendants, whether immediate or not, of the first generation of microorganisms.

Within this specification, including the claims, the phrase “continuing generation of microorganisms,” and the like, is defined to include a generation of microorganisms which are reproductive descendants, whether immediate or not, of the subsequent generation of microorganisms.

Within this specification, including the claims, the phrase “micro-organism quantity” is defined to include the microorganisms of the biological composition, including all reproductive descendants. Further, it includes any and all other material which may be present with the microorganisms as they are mixed together with other materials to form the biological composition. These materials may include inert carrier materials as well as any other materials.

Within this specification, including the claims, the term “vegetation,” and the like, is defined to include all plants and other flora.

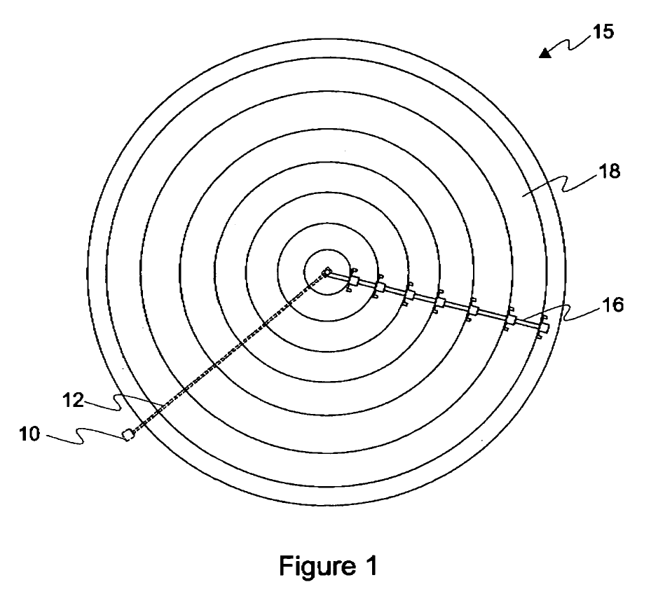

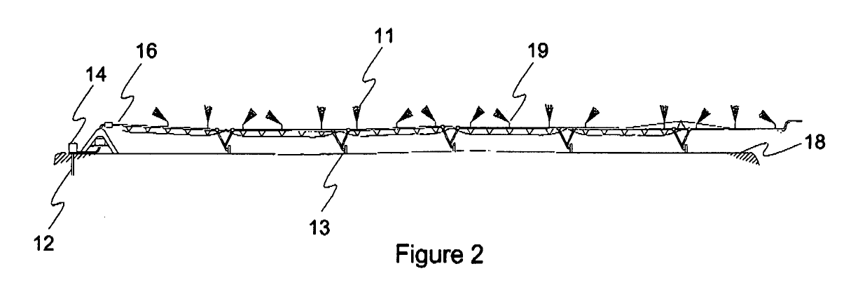

FIGS. 1 and 2 illustrate a system configured to introduce microorganisms into a plant ecosystem according to one embodiment of the invention. There is a tank, or container 10, connected to a feed pipe 12, containing irrigation water and extending underground, of an irrigation system 15 configured to irrigate a plant ecosystem 18. The irrigation system 15 further includes a junction 14 connecting the feed pipe 12 to the irrigation pipe 16. The irrigation pipe 16 includes sprinklers 19 configured to distribute fluid 11 over the plant ecosystem 18. Further, there are wheels 13 configured to permit displacement of the irrigation pipe 16 about the plant ecosystem 18.

In operation, microorganisms are generated, or regenerated, as products and further generations of a biological composition, in a liquid mixture in the container 10. The feed pipe 12 may supply water to the container 10 as needed. The liquid mixture is introduced into the feed pipe 12 at a known rate or at known portions per elapsed time. Preferably, the rate at which the liquid mixture is introduced into the feed pipe 12 does not exceed the rate at which the liquid mixture is generated by the container 10. For example, where the container generates, or regenerates, 100 gallons of liquid mixture per day, it is preferable that the rate at which the liquid mixture is introduced, or injected, into the feed pipe does not exceed 100 gallons per day, regardless of whether the rate is constant throughout a twenty-four hour period. The liquid mixture portions mix with the irrigation water as the irrigation water flows through the feed pipe 12, through the junction 14, through the irrigation pipe 16, through the sprinklers 19, which sprinklers distribute the combined fluids 11 over the plant ecosystem 18.

Micro-organism generation, and/or regeneration, consumes nutrients. Further, generations of microorganisms tend to mutate, or change, from strains originally introduced. Therefore, preferably, nutrients and original strains of microorganisms are introduced into the container as needed. For example, biological compositions, preferably in pre-made packets, including a nutrient quantity and a quantity of original strain(s), or micro-organism quantity, are preferably placed within the container on a regular schedule, such as once a week.

In operation of one embodiment, the biological composition may provide a two stage rich source of food and other nutrients for the quantity of microorganisms. The first stage permits rapid reproduction of the micro-organism. This beneficially permits the micro-organism to predominate other competing microorganisms and creates a large quantity of microorganisms in a short period of time, which then may be dispersed into a plant ecosystem. The second stage facilitates maintenance of the micro-organism population as portions are extracted and dispersed into a plant ecosystem. During both stages, important nutrients are supplied by the biological composition to the reproducing micro-organism population.

Additionally, in operation of one embodiment, the two stage rich source of food is configured to last for a reproduction period, with the materials providing the first stage being substantially consumed before the expiration of the reproduction period. For example, the biological composition may be configured to last for a week, with the first stage configured to be substantially consumed within 48 hours. In another example, the biological composition may be configured to last 4 days, with the first stage configured to be substantially consumed within 24 hours.

One skilled in the art would know that by varying the proportions and total amounts of the materials comprising the ingredients of the two stages and the initial micro-organism content of the biological composition, one may adjust the biological composition to generally conform the reproduction period to any reasonable desired period of time. Also, it is not necessary that the reproduction period be known to the manufacturer, or that it be designated at all, merely that there be a period of time to which the composition relates as described herein.

Also, in operation of an embodiment, oxygen is introduced to a liquid mixture containing microorganisms in a container. This may be accomplished by injection, bubbling, interface exchange, or any other method known in the art for providing access to oxygen within a liquid. The source of oxygen may be normal air.

Still yet, in operation of one embodiment, portions of a liquid mixture containing microorganisms in a container are extracted from the container. This may be accomplished with an outlet, preferably coupled to a lower portion of the container to avoid clogging with any surface skins formed on the top surface of the liquid mixture in the container.

In addition, the nutrient quantity preferably includes a food source easily utilized by the microorganisms, or short-term nutrient, to promote rapid generation of microorganisms, in particular, rapid generation of the original strain. Additionally, the nutrient quantity preferably includes a long-lasting food source, or long-term nutrient, configured to nourish the microorganisms after the short-term food source depletes. Also, the nutrient quantity preferably includes a quantity of other nutrients, or supplement nutrients. The nutrient quantity may include but is not limited to vitamins, minerals, enzymes, amino acids, protein compositions, starches, fibers, carbohydrates, sugars, growth media, proteins, chelating agents, complexing agents, sequestering agents, and other materials useful in nourishing microorganisms and plants.

In particular, it is preferred that the micro-organism quantity includes a micro-organism(s) characterized by the ability to enhance vegetation growth, preferably in a plant dominated ecosystem such as a field of human cultivated plants. Examples of beneficial microorganisms include, but are not limited to, bacteria, yeasts, protozoa, actinomycites, fungi, and nematodes. It is preferred that the micro-organism quantity includes a micro-organism(s) characterized by the ability to produce cellulase enzymes, to convert nutrients to a form usable by plants, to loosen soil, to enhance water retention in soil, to combat parasitic organisms, and/or to otherwise enhance soil vitality, plant vitality, crop production, plant health, and/or crop production efficiency. Additionally, the microorganisms may be aerobic bacteria. The micro-organism(s) may include, but is not limited to bacillus subtilis, bacillus lichenformis, bacillus cereus, bacillus megaterium, fluorescent pseudomonas, azobacter, cellulase enzyme producing bacteria, yeasts, sub-cultures thereof, and combinations thereof.

Still further, it is preferred that the micro-organism(s) in the micro-organism quantity be included in sufficient quantities to predominate other microorganisms which may use the biological composition to reproduce. “Other microorganisms,” as used in the previous sentence, may include microorganisms present in a container, in a water supply feeding into a container, strains of similar microorganisms which may have mutated from an original strain related to, or identical to, the microorganisms present in the micro-organism quantity.

Still, in particular, it is preferred that the short-term nutrient include ingredient(s) characterized by the ability to provide a quick and ready source of nourishment for the microorganisms of the micro-organism quantity. Preferably, this may include, but is not limited to, hydrolyzed collagen, bone meal, blood meal, carbon skeleton molecules, sugars, carbohydrates, folic acid, organic acid, soy protein, peptone treated biological matter (such as peptone treated animal carcasses or peptone treated plant matter), other easily consumed materials and combinations thereof. Preferably, the short-term nutrient is present in the biological composition in sufficient amounts to provide for rapid reproduction of the micro-organism quantity and its further generations for a reproduction period.

Further, in particular, it is preferred that the long-term nutrient include ingredients(s) characterized by the ability to provide a stable, lasting (as compared to the short-term nutrient and/or the intended period of replacement of the biological composition packets) source of nourishment for the microorganisms of the micro-organism quantity. Preferably, this may include, but is not limited to, wheat starch, soy flour, molasses, processed or raw animal and/or plant matter, other slowly consumed proteins, fibers, starches, fats and carbohydrates and combinations thereof. Preferably, the long-term nutrient is present in the biological composition in sufficient amounts to provide for continued reproduction of the micro-organism quantity and its further generations for a reproduction period after the short-term nutrient quantity is substantially consumed.

Again, in particular, it is preferred that the supplement nutrient include ingredient(s) characterized by the ability to provide for the variety of nourishment needs of the microorganisms of the micro-organism quantity. It is preferred that the supplement nutrient at least provides for at least one the non-energy source needs of the microorganisms of the micro-organism quantity. Preferably, the ingredient(s) of the supplement nutrient may include, but is not limited to, food-grade proteins; vitamins; inorganic salts; amino acids; growth media; minerals such as phosphate, potassium, calcium, sulfur, cobalt, copper, iron, magnesium, sodium, manganese, and zinc; humate and/or humic acids; enzymes; chelating, complexing, and/or sequestering agents, with or without associated molecules; and combinations thereof. Preferably, the supplement nutrient is present in the biological composition in sufficient amounts to nourish the micro-organism quantity and its further generations for a reproduction period.

Preferably, the biological composition is in a dry form wherein the biological composition may be stored for a time with the microorganisms in an inactive state. Preferably, the biological composition is a package. Preferably, the biological composition may be powder, granules, or a pressed cake. In addition, the biological composition is preferably configured to aid, or induce, generation or regeneration of a quantity of microorganisms, preferably in a container or system configured to introduce microorganisms into a plant ecosystem. Also, preferably, the biological composition is configured to introduce, or reintroduce, an early generation of the microorganisms into the container or system. “Early generation” means that the microorganisms are not substantially mutated from the desired species, strains, and/or characteristics.

### EXAMPLE ONE

There is a dry micro-organism amount which includes base soil bacteria; for example, the product known under the brand name Soil Response™. The product known as Soil Response™, is attributed to SafeWaze, 7411 N. Tryon Street, Charlotte, N.C. 28213. The product known as Soil Response™ is a mixture of active hydrocarbon oxidizing, natural single-cell organisms, specifically for use on soil including, but is not limited to Pseudomonas Fluorescent, Azotobacter, as well as Cellulase enzymes producing bacteria. The microbes are contained in an inert preparation of a natural absorbent, which has no chemical impact.

There is a dry nutrient amount which includes a micro-nutrient supplement, a long-term food source and a short term food source. The micro-nutrient supplement is manufactured by the organization having the trademark SafeWaze™ at 7411N. Tryon Street, Charlotte, N.C. 28213. It includes a blend of food-grade proteins, vitamins, inorganic salts, and growth media—intended as a supplementary food supply for microorganisms. The micro-nutrient package may include, but is not limited to, minerals and nutrients including phosphate, potassium, calcium, sulfur, cobalt, copper, iron, magnesium, and zinc, as well as proteins. The long-term food source is produced under the label DRI-MOL®, which is a dry molasses product manufactured by the organization having the trademark ADM found in Stanley, Wis. 54768. The ingredients include molasses, wheat Starch, calcium strearate, soy flour, and lecithin. The short-term food source is hydrolyzed collagen, or HC, of type GCP-1000 which is manufactured by the organization known as Nitta Gelatin NA, INC., 201 W. Passaic Street, Rochelle Park, N.J. 07662.

The composition amounts are detailed below, with a period of usage of seven days and a fresh clean out at the beginning of each season.

### Biological Package Composition and Application Usage:

| | | | Applied into | |
|---|---|---|---|---|
| | | | Container on | |
| | Density | | Days 1, 7, 14, 21 | |
| Soil Response TM | 6.25 | ounces/cup | 0.625 | cups |
| Micro-Nutrient Supplement | 6 | ounces/cup | 0.125 | cups |
| DRI-MOL ® | 5.5 | ounces/cup | 2.5 | cups |
| Hydrolyzed Collagen HC | 3.25 | ounces/cup | 0.75 | cups |

### EXAMPLE TWO

| | Approximate Percent by Volume |
|---|---|
| Dry Bacteria Culture | 5% |
| Yeast | 1% |
| Soybean Protein | 18.75% |
| Nutrient Mineral Mix | 18.75% |
| Humate | 18.75% |
| Granulated Sugar | 18.75% |
| Flour | 18.75% |

The nutrient mineral mix included crude protein, crude fat, salt, calcium, chlorine, magnesium, phosphorous, potassium, sodium, sulfur, cobalt, copper, iodine, iron, manganese, and zinc.

### EXAMPLE THREE

Examples of materials which are includable in embodiments include a variety of different biological and food mixes including soy protein (used for animal feed supplements), mineral mixes (used in animal nutrition), protein packages (used in body building and dietary supplement), flours, sugar, raw molasses, yeast, various enzymes used in waste treatment, laundry soaps, and the product sold under the mark Oxy Clean. Also included are various combinations of products including the product sold under the marks ViBasic, Xcite and ViPlex from the organization under the mark Vitech Industries. Further examples include the product under the mark Esp333, both liquid and dry, as well as a protein feed supplement from the organization under the mark Bio-Kinetics, soil stimulant from the organization known as Fertile Earth, along with various combinations of fertilizers.

### EXAMPLE FOUR

Using flood irrigation, a farmer raised 25% more alfalfa hay than his prior average using 6.5 gallons of a product of a biological composition, as claimed and described, dripped into the first irrigation, 15 gallons in his second irrigation, and 30 gallons in third. On two fields, the treatment so improved water retention that only two irrigations were required to achieve the same yield increase.

### EXAMPLE FIVE

Using pivot irrigation, a farmer farms 80 pivots. Most of the pivots are 125-acre pivots, with a few going up to 160 acres. One pivot is 540 acres. Due to the size of the 540-acre pivot and the time it takes for one rotation of the 540 acre pivot, the farmer was having problems with it drying out. This affected yields and hay quality. The farmer started applying a product of a biological composition dispersed as claimed through the pivot at a rate of 400 gallons per day. By the time of the first cutting, the hay stand was thicker and the soil was moist. Throughout the course of the year, the fields continued to see soil, yield, and quality improvements.

### EXAMPLE SIX

Using pivot irrigation, a farmer had a pivot on a hillside where the high side of the field would not retain water and the alfalfa growth was always shorter. The farmer had tried other products on the field with little results. Use of the products of the biological composition commenced during the heat of the summer when water problems were at a peak. After just a few weeks of using one of the products of the biological composition, at the rate of 120 gallons per day, growth on the high side of the field was as good as the rest of the field and yielding the same.

### EXAMPLE SEVEN

A dry powdered micro-organism amount, or quantity, was mixed with a dry powdered plurality of nutrients, or nutrient quantity, according the following:

| | | | WEIGHT |
|---|---|---|---|
| | CUPS | WEIGHT | PERCENTAGE |
| COMPONENT | PER MIX | PER MIX | PER MIX |
| 1. Micro-organism/ | 2.5 | 1.20 | 16% |
| Bacteria | | | |
| 2. Blue Nutrient | 0.25 | 0.20 | 3% |
| 3. Collagen | 7 | 1.11 | 15% |
| 4. Dry Molasses | 6 | 3.33 | 45% |
| with Starch Base | | | |
| 5. Granulated Sugar | 3 | 1.04 | 14% |
| 6. Yeast | 0.04 | 0.03 | Less than 1% |
| 7. Blood Meal | 0.5 | 0.20 | 3% |
| 8. Bone Meal | 0.5 | 0.31 | 4% |
| Total | 19.29 | 7.43 | 100% |

The micro-organism quantity was from a 35 pound bucket of the product known as Remediact™, by Envirologic, Wyncote, Pa. However, one skilled in the art would understand that the micro-organism quantity may include, but is not limited to, the following microorganisms: bacillus subtilis, bacillus lichenformis, bacillus cereus, bacillus megaterium, fluorescent pseudomonas, azobacter, cellulase enzyme producing bacteria, yeast, and/or sub-cultures thereof. In addition, the 35 pound bucket of the product known as Remediact™, by Envirologic, Wyncote, Pa., included Blue Nutrient, a component of the nutrient quantity.

In addition, the nutrient quantity of the experiment included collagen, dry molasses, granulated sugar, yeast, blood meal, and bone meal. The collagen was from a 40 pound bag of hydrolyzed pork collagen, by Geltex (North Central Companies), Minnetonka, Minn. Collagen provides and added benefit as a high protein, containing up to 95% protein, thereby providing and energy source for the microorganisms for an extended period of time.

The dry molasses was from a 50-pound bag of the product known as Dry-Mol™, by Chicago Sweeteners, Des Plaines, Ill. Accordingly, the dry molasses was dispersed within a matrix of a granular starch base. However, one skilled in the art would understand that any molasses sprayed on, or dispersed within a matrix of, a grain residue carrier, or starch substrate, may be used. Beneficially, the use of dry molasses in the composition with microorganisms of the product Remediact™ results in stimulation of the beneficial microorganisms. In particular, molasses is a sweet syrup, or carbohydrate, that is used as a soil amendment to feed and stimulate micro-organism metabolisms. Molasses contains carbon, sulfur, potash, and many trace minerals. As a result, molasses provides food for microorganisms and is a quick source of energy for beneficial microbes thereby creating greater nature fertility. Molasses syrup may be dried, but dried molasses is often very difficult to acquire, and very difficult to work with, as its viscosity is easily affected by humidity, causing it to become sticky in wet environments. Accordingly, dry molasses, or molasses blended with a carrier such as a grain or soy, may be blended into a fine powder for even mixing and application.

The granulated sugar and yeast may be provided by any grocery store, such as Sam's Club, by Sam's West, Inc., Bentonville, Ark. For example, the granulated sugar may be C&H pure cane sugar by C&H Sugar Company, Inc., Crockett, Calif., and the yeast may be Fleischmann's Yeast, by ACH Food Companies, Inc. of Memphis, Tenn. Accordingly, one skilled in the art would understand that sugar refers to any monosaccharide or disaccharide. Monosaccharides (also called “simple sugars”), such as glucose, store chemical energy which biological cells convert to other types of energy.

Further, one skilled in the art would appreciate that, along with bacteria, yeast or fungi, are the major decomposers in most terrestrial ecosystems, and, therefore, play a critical role in biogeochemical cycles and in many food webs. As a result, yeasts are often indispensable in nutrient cycling, especially as saprotrophs and symbionts, degrading organic matter to inorganic molecules, which can then re-enter anabolic metabolic pathways in plants or other organisms. Mycorrhizal symbiosis between plants and yeast is of significant importance for plant growth and persistence in many ecosystems, as over 90% of all plant species engage in some kind of mycorrhizal relationship with fungi and are dependent upon this relationship for survival. The mycorrhizal symbiosis often increases the plant's uptake of inorganic compounds, such as nitrate and phosphate from soils having low concentrations of these key plant nutrients. In some mycorrhizal associations, the fungal partners may mediate plant-to-plant transfer of carbohydrates and other nutrients. Lichens are formed by a symbiotic relationship between algae or cyanobacteria and fungi, in which individual photobiont cells are embedded in a tissue formed by the fungus. As in mycorrhizas, the photobiont provides sugars and other carbohydrates, while the fungus provides minerals and water.

The blood meal included nitrogen, phosphorus, and potassium. In particular, the blood meal was dried, and powdered blood was used as a high-nitrogen fertilizer. For example, one skilled in the art would understand that the blood meal may be High Yield blood meal such as 12-0-0. Beneficially, blood meal is one of the highest non-synthetic sources of nitrogen. In addition, blood meal is completely soluble and may be mixed with water for use as a liquid fertilizer. However, if over-applied, blood meal can burn plants due to excessive ammonia.

The bone meal included nitrogen, phosphorus, potassium, and calcium. In particular, the bone meal was a mixture of crushed and coarsely ground bones often used as an organic fertilizer. For example, one skilled in the art would understand that the bone meal may be High-Yield blood meal such as 0-10-0. Beneficially, bone meal provides an excellent source of phosphorous, which is a slow-release fertilizer.

In operation of one embodiment of the invention, a biological composition is mixed within a container wherein the biological composition includes a dry powdered micro-organism quantity; and a dry powdered nutrient quantity. The dry powdered nutrient quantity includes one or more nutrients selected from the group consisting of: blue nutrient, collagen, dry molasses dispersed within a matrix of a granular starch base, sugar, yeast, blood meal, and bone meal.

The dry powdered micro-organism quantity and dry powdered nutrient quantity are then mixed with water, separated from the dry powder, in the container. Oxygen is then introduced into the biological composition, and the biological composition is circulated. Next, biological composition portions are extracted from the container at an average rate not significantly greater than an average regenerative rate of the biological composition. Accordingly, a quantity of microorganisms remains in the container for regeneration and growth. Then, the biological composition portion is introduced into irrigation water in an irrigation system, and plants are irrigated with the combined biological composition portion and irrigation water. Finally, an additional quantity of dry powdered microorganisms and nutrients is added to the powdered biological composition in the water, thereby replenishing and enhancing micro-organism vitality, growth, and reproduction.

Advantageously, the nutrients enable feeding, reproduction, and growth of microorganisms prior to their application, or dispersal, to soil and/or plants for enhancing plant growth and/or water retention. Moreover, rather than applying nutrients and microbes separately, one at a time, and/or in layers, mixing microorganisms with nutrients prior to dispersal gives the microorganisms' metabolisms a jump start in stimulating biological activity. Beneficially, the overall amount of the composition, which must be dispersed, may be decreased, as well as the number of applications which may be required. As a result, a single application of the composition is all that may be required for plant production. Furthermore, the weight percentages of the composition, as claimed and described, are preferred for enabling optimal growth and reproduction of soil microbes.

In addition, mixing microorganisms with nutrients prior to dispersal to stimulate biological activity provides immediate results. Further, the biological composition may be easily applied through irrigation systems. For example, the biological composition may be mixed with water in an irrigation system and sprayed on plants and/or soil thereby providing an even spread of nutrients and/or soil microbes for even growing conditions.

It is understood that the above-described preferred embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claim rather than by the foregoing description. All changes, which come within the meaning and range of equivalency of the claims, are to be embraced within their scope.

It is also understood that the words “irrigate” and “irrigation” are not limited to one, or several means, to provide water, such as flood irrigation or pivot irrigation. Unless otherwise indicated, the terms “irrigate” and “irrigation” and similar terms are intended to indicate any means or act of providing with water or other similar fluid.

In addition, although the figures illustrates a particular irrigation system and growth container, it is understood that not all details of the configuration and/or conjunction are shown. Also, it is envisioned that the invention may be adapted to couple with any system for dispersing water into a plant ecosystem, or that the invention may be an integral part of such system.

Additionally, although the figures and examples illustrate specific compositions, it is understood that the scope of the invention is to be determined by the claims and not by the specific examples.

It is also envisioned that one embodiment may include one material as a short-term nutrient while another may include that same material as a long-term nutrient, depending on the characteristics of the microorganisms in the micro-organism quantity and/or the intended period of packet replacement.

One skilled in the art would know that, although the invention is sometimes expressed as a mixture of quantities, it is not necessary that the quantities be distinct. For example, the same ingredient forming the short-term nutrient quantity may also supply at least some of the supplement nutrient quantity, as may be the case where a short-term nutrient quantity includes hydrolyzed collagen.

It is expected that there could be numerous variations of the design and/or configuration of embodiments of this invention. An example is that the biological composition may include additional materials beyond those disclosed. These materials may be carrier materials, inert materials, granulation materials, caking materials, or may or may not, serve another function such as a beneficial function related to the packaging, shipping, storing, manufacturing, usage, and/or compliance with authority needs.

Finally, it is envisioned that the embodiments of the invention may be constructed of a variety of materials. It is envisioned that the embodiments of the invention may include materials, not herein disclosed but which may be known in the art, having properties substantially similar to those that have been disclosed.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly, and use, may be made without departing from the principles and concepts of the invention as set forth in the claims.

What is claimed is:

1. A biological composition for generating and feeding microorganisms that are intended for distribution in an agricultural system, comprising:
a micro-organism quantity, including a first generation of microorganisms; and
a nutrient quantity, configured to provide nutrients to the micro-organism quantity to sustain reproduction of the micro-organism quantity for a reproduction period, including:
a granular starch base;
dry molasses dispersed within a matrix of the granular starch base; and
one or more nutrients selected from the group consisting of: blue nutrient, collagen, sugar, yeast, blood meal, and bone meal;
wherein the micro-organism quantity and the nutrient quantity are a dry powder.

2. The biological composition of claim 1, further comprising water separate from the dry powder.

3. The biological composition of claim 1, wherein the micro-organism quantity comprises a micro-organism type selected from the group consisting of: bacillus subtilis, bacillus lichenformis, bacillus cereus, bacillus megaterium, fluorescent pseudomonas, azobacter, cellulase enzyme producing bacteria, yeast, and sub-cultures thereof.

4. The biological composition of claim 1, wherein the micro-organism quantity comprises from about 1% to about 25% of the biological composition by weight, and the nutrient quantity comprises from about 75% to about 99% of the biological composition by weight.

5. The biological composition of claim 4, wherein the nutrient quantity comprises:
about 3% of the biological composition by weight blue nutrient;
about 15% of the biological composition by weight collagen;
about 45% of the biological composition by weight dry molasses and granular starch base;
about 14% of the biological composition by weight sugar;
less than 1% of the biological composition by weight yeast;
about 3% of the biological composition by weight blood meal; and
about 4% of the biological composition by weight bone meal.

6. The biological composition of claim 5, wherein the collagen comprises hydrolyzed pork collagen.

7. The biological composition of claim 5, wherein the sugar comprises granulated sugar.

8. A method for generating and feeding microorganisms that are intended for distribution in an agricultural system, comprising the following steps:
providing a container configured to house a liquid mixture;
providing water within the container;
providing a dry powdered biological composition in the water, wherein the dry powdered biological composition includes:
a micro-organism quantity, including a first generation of microorganisms; and
a nutrient quantity, configured to provide nutrients to the micro-organism quantity to sustain reproduction of the micro-organism quantity for a reproduction period, including:
a granular starch base;
dry molasses dispersed within a matrix of the granular starch base; and
one or more nutrients selected from the group consisting of: blue nutrient, collagen, sugar, yeast, blood meal, and bone meal;

9. The method of claim 8, wherein the micro-organism quantity comprises a micro-organism type selected from the group consisting of: bacillus subtilis, bacillus lichenformis, bacillus cereus, bacillus megaterium, fluorescent pseudomonas, azobacter, cellulase enzyme producing bacteria, yeast, and sub-cultures thereof.

10. The method of claim 8, wherein the micro-organism quantity comprises from about 1% to about 25% of the biological composition by weight, and the nutrient quantity comprises from about 75% to about 99% of the biological composition by weight.

11. The method of claim 10, wherein the nutrient quantity comprises:
about 3% of the biological composition by weight blue nutrient;
about 15% of the biological composition by weight collagen;
about 45% of the biological composition by weight dry molasses and granular starch base;
about 14% of the biological composition by weight sugar;
less than 1% of the biological composition by weight yeast;
about 3% of the biological composition by weight blood meal; and
about 4% of the biological composition by weight bone meal.

12. The method of claim 8, further comprising the steps of:
introducing oxygen into the biological composition; and
circulating the biological composition.

13. The method of claim 12, further comprising the step of extracting biological composition portions from the container at an average rate not significantly greater than an average regenerative rate of the biological composition.

14. The method of claim 13, further comprising the steps of:
introducing the biological composition portion into irrigation water in an irrigation system; and
irrigating plants with the combined biological composition portion and irrigation water.

15. The method of claim 11, wherein the collagen comprises hydrolyzed pork collagen.

16. The method of claim 13, further comprising repeating the step of providing a dry powdered biological composition in the water.

17. A biological composition for generating and feeding microorganisms that are intended for distribution in an agricultural system, comprising:
from about 1 weight percent to about 25 weight percent of a dry powdered micro-organism quantity, including a first generation of microorganisms; and
from about 75 weight percent to about 99 weight percent of a dry powdered nutrient quantity, configured to provide nutrients to the micro-organism quantity to sustain reproduction of the micro-organism quantity for a reproduction period, including:
a granulated starch base;
dry molasses dispersed within a matrix of the granular starch base; and
one or more nutrients selected from the group consisting of: blue nutrient, collagen, sugar, yeast, blood meal, and bone meal;
wherein the micro-organism quantity and the nutrient quantity are a dry powder; and
water separate from the dry powder.

18. The biological composition of claim 17, wherein the micro-organism quantity comprises a micro-organism type selected from the group consisting of: bacillus subtilis, bacillus lichenformis, bacillus cereus, bacillus megaterium, fluorescent pseudomonas, azobacter, cellulase enzyme producing bacteria, yeast, and sub-cultures thereof.

19. The biological composition of claim 17, wherein the nutrient quantity comprises:
about 3% of the biological composition by weight blue nutrient;
about 15% of the biological composition by weight collagen;
about 45% of the biological composition by weight dry molasses and granular starch base;
about 14% of the biological composition by weight sugar;
less than 1% of the biological composition by weight yeast;
about 3% of the biological composition by weight blood meal; and
about 4% of the biological composition by weight bone meal.

20. The biological composition of claim 19, wherein the collagen comprises hydrolyzed pork collagen and the sugar comprises granulated sugar.
